SEQUENCE LISTING (6 sequences)

SEQ ID NO: 1 - DNA, 90 bp, Artificial Sequence
  Synthetic Construct
tttccaagtaagcaataacgtcagctctttcttgtggcttcttcataccagcgaaagacatcttagtacctggcatgaacttctttgggt

SEQ ID NO: 2 - DNA, 87 bp, Artificial Sequence
  Synthetic Construct
acccaaagaagttcatgccaggtactaagatgtctttcgctggtatgaagaagccacaagaaagagctgacgttattgctttggaaa

SEQ ID NO: 3 - DNA, 166 bp, Artificial Sequence
  Synthetic Construct
ctgcaggcgccagcgtggaccatcaagtagtaatgaacgcacggacgaggacatcatagagattacacctttatccacagttctcggtctaacgcagcagtcagtgtatcagcaccagcatccgtagtgagtcttcagtgtctgctccaggatcgtggcgctgcag

SEQ ID NO: 4 - DNA, 26 bp, Artificial Sequence
  Synthetic Construct
cgccagcgtggaccatcaagtagtaa

SEQ ID NO: 5 - DNA, 26 bp, Artificial Sequence
  Synthetic Construct
cacgatcctggagcagacactgaaga

SEQ ID NO: 6 - DNA, 30 bp, Artificial Sequence
  Synthetic Construct
ctgcgttagaccgagaactgtggataaagg

(19) United States
(12) Patent Application Publication
(10) Pub. No.: US 2013/0244894 A1
(43) Pub. Date: Sep. 19, 2013

(54) NUCLEIC ACID-BASED AUTHENTICATION CODES

(71) Applicant: AUTHENTIFORM TECHNOLOGIES, LLC, Chapel Hill, NC (US)

(72) Inventor: Thomas J. Mercolino, Chapel Hill, NC (US)

(73) Assignee: AUTHENTIFORM TECHNOLOGIES, LLC, Chapel Hill, NC (US)

(21) Appl. No.: 13/796,054

(22) Filed: Mar. 12, 2013

Related U.S. Application Data
(60) Provisional application No. 61/610,244, filed on Mar. 13, 2012.

(51) Int. Cl. C12Q 1/68; G01N 33/58

(52) U.S. Cl. CPC C12Q 1/6834; G01N 33/582
USPC 506/9; 436/501; 435/6.11

(57) ABSTRACT

This invention relates to a nucleic-acid based product authentication by determining authentication codes comprising target nucleic acids using oligonucleotide probes immobilized on particulate and non-particulate substrates. The presence of the authentication code is determined using detection methods, such as flow cytometric methods, capable of particle discrimination based on the light scattering or fluorescence properties of the particle, or by spatial resolution of oligonucleotides immobilized at specific loci on a substrate. Target-correlated fluorescence signal, originating from a target nucleic acid hybridized to substrate-immobilized oligonucleotide is determined as an indicator of the presence of the authentication code.

### CROSS-REFERENCE TO RELATED APPLICATIONS

This application claims priority to U.S. Provisional Patent Application No. 61/610,244, entitled Nucleic Acid-Based Authentication Codes, filed on Mar. 13, 2012, the contents of which are hereby incorporated by reference in their entirety.

### FIELD OF THE INVENTION

This invention is directed to nucleic acid based product authentication by determining the presence of target nucleic acids. In particular, the present invention provides methods for simplifying the detection process so that the authenticating nucleic acid can be detected in situ in the formulation of the product, on the surface of a product, or on product packaging, without sequencing. It also provides methods for achieving higher complexity, and hence more tamperproof, authentication codes comprising a combination of nucleic acids and taggants and marking locations in relatively small sets to allow for relatively large numbers of unique codes.

### BACKGROUND OF THE INVENTION

Counterfeit items continue to pose a significant and growing problem with consumer packaged goods, especially for established brands. Because of their intrinsic capability to carry diverse coded information, nucleic acids have been used to provide a secure, cost effective and forensic method to help companies protect their intellectual property and brand. Typically, nucleic acids are applied to the commercial article by means that stabilize it to manufacturing processes, then the nucleic acid can be sequenced to verify the product's authenticity. Unique nucleic sequences must be synthesized for each coding identifier.

Likewise, microparticulate taggants have been used as means for authenticating products. With such taggants, combining tags that have different detectable physical properties, wherein each combination of properties is used as an encoding bit to create codes. Similarly to nucleic acids, unique taggants with unique combinations of physical properties must be manufactured in order to increase the number and complexity of possible codes.

The detection of nucleic acids is widely employed for determining the presence and copy number of specific genes and known sequences. An important characteristic of nucleic acids is their ability to form sequence-specific hydrogen bonds with a nucleic acid having a complementary nucleotide sequence. This ability of nucleic acids to hybridize to complementary strands of nucleic acids has been used to advantage in what are known as hybridization assays, and in DNA purification techniques. In a hybridization assay, a nucleic acid having a known sequence is used as a probe that hybridizes to a target nucleic acid having a complementary nucleic acid sequence. Labeling the probe allows detection of the hybrid and, correspondingly, the target nucleic acid.

Because of their intrinsic capability to carry diverse coded information, nucleic acids have been used to provide a secure, cost effective and forensic method to help companies protect their intellectual property and brand. U.S. Pat. No. 5,139,812 discloses the use of nucleic acid sequences in ink for identifying an object with a probe. U.S. Pat. No. 5,451,505 uses nucleic acids as taggants. Such techniques also are not readily perceptible without the aid of special equipment, which develop the presence of such markers. US20050112610 812 discloses the use of nucleic acid sequences for identifying textiles. However, each unique code has required that a separate, unique target nucleic acid sequence be synthesized, increasing cost and decreasing practicality of the methods. Moreover, complex and expensive sequencing analysis has typically been required to identify such unique sequences.

Certain of the above limitation have been overcome in Identif's Bio-Molecular Marker Covert system, which is based on a synthetic target nucleic acid sequence supplied as an ink. The product's label is imprinted with a customer-specific ink, which can be identified in the field by activating the marking with a pen that contains the mating strand. One of the half strands of DNA is accompanied by a “molecular beacon” fluorophore. When unmatched, the fluorophore is not visible. But when matched with a mating strand, it opens and the fluorophore becomes detectable. The signal in this invention does not require that either strand be synthesized incorporating costly molecular beacon technology. Additionally, the methods of this invention do not require synthesis of separate, unique target nucleic acid sequences for each customer-specific code, as is the case with Identif's method.

Applied DNA Sciences offers the SigNature™ Program based on APDN's plant-derived DNA sequences. Botanical DNA is encrypted into inks, paper, thread, holograms and other mediums, or printed in a botanical DNA SigNature logo that highlights the word “Nature”. The logo has been designed to contain embedded botanical DNA, for overt detection and forensic authentication purposes. For real-time detection, APDN offers a proprietary SigNature DNA detection pen that is applied over the DNA-embedded SigNature logo, prompting a reversible color change. No details have been uncovered relating to the chemistry of detection, but the APDN method is subject to the same limitations as those identified for Identif's.

This invention describes methods for simplifying the detection process so that the authenticating nucleic acid can be detected in situ on the product without sequencing. It also provides methods to achieve higher complexity, and hence more tamperproof, authentication codes comprising a combination of nucleic acids and taggants and marking locations in relatively small sets to allow for relatively large numbers of unique codes. The requirement for detector oligonucleotide to match target nucleic acid provides for “lock & key” assurance.

### SUMMARY OF THE INVENTION

In one aspect, the present invention relates to a method for authenticating a product by determining the presence of a target nucleic acid in the formulation of the product, on the surface of a product, or on the product packaging, comprising the steps of:

a) associating an authentication code comprising the target nucleic acid with the article to be authenticated; b) contacting the article with an oligonucleotide probe comprising a nucleic acid sequence complementary to at least a portion of the target nucleic acid; and c) determining the presence of the authentication code by contacting the duplex heterodimer formed by said target nucleic acid and oligonucleotide probe to a reporter compound which is capable of binding to duplex nucleic acid and which upon binding or being bound thereto is capable of producing a detectable signal. Association may be achieved preferably by immobilization within the formulation of a solid product (e.g., paper), on the surface of a product, or on product packaging

Optionally, steps b and c are performed sequentially, or preferably, at the same time. A detectable signal, or signal change correlates to immobilized target hybridized to oligonucleotide probe and is thereby an authentication signal. The methods of this embodiment are particularly well suited to detection of the target nucleic acid with a pen that contains both oligonucleotide probe and an intercalating dye. The requirement for detector oligonucleotide, that is, the oligonucleotide probe to match target nucleic acid provides for “lock & key” assurance.

In one embodiment, the method includes an authentication pouch comprising the authentication which comprises the target nucleic acid(s) and/or microparticles having distinctly measurable properties.

In another aspect, the present invention relates to a method for authenticating a product by determining the presence of an authentication code comprising at least two target nucleic acid sequences and comprising the steps of: a) immobilizing each target nucleic acid to the product to be authenticated at a discernable location on the article; b) contacting said locations with at least two oligonucleotide probes each comprising nucleic acid sequences complementary to at least a portion of one of the target nucleic acids, and c) determining the presence of the authentication code by contacting the duplex heterodimer formed by said target nucleic acids and oligonucleotide probes to a reporter compound which is capable of binding to duplex nucleic acid and which upon binding or being bound thereto is capable of producing a detectable signal.

Optionally, steps b and c are performed sequentially, or preferably, at the same time. Optionally, immobilization is performed in a specified morphology or symbology wherein revealing the presence of the target oligonucleotide also reveals a specific shape or symbol and/or size at each location. A detectable signal, or signal change at said discernable location correlates to immobilized target hybridized to oligonucleotide probe. Combining location with morphology and size allows very few unique target nucleic acid sequences to be synthesized in order to achieve very high complexity codes. For example, selecting 2 of the characters A-Z and numbers 0-9 (36 possibilities) for each of two locations, and printing each location with either of 2 possible target nucleic acid sequences and in 3 possible discernable sizes yields 7776 possible codes from just these two sequences. Again, the methods of this embodiment are equally well suited to detection with a pen system and provides for “lock & key” assurance, as above.

In another embodiment of the present invention, the method for authenticating an article or product by determining the presence of a target nucleic acid comprises the steps of: a) immobilizing the target nucleic acid to a substrate, wherein the substrate is a particle that is capable of scattering electromagnetic radiation of wavelength greater than or equal to about 200 nm; or comprises a distinct first compound or plurality of compounds capable of producing a distinct fluorescence signal corresponding to the particle; or capable of said scattering and producing said fluorescence signal; b) associating said substrate with the article to be authenticated; c) contacting the substrate to an oligonucleotide probe comprising a nucleic acid sequence complementary to at least a portion of the target nucleic acid, and d) contacting the duplex heterodimer formed by said target nucleic acid and oligonucleotide probe to a reporter compound which is capable of binding to duplex nucleic acid and which upon binding or being bound thereto is capable of producing a detectable signal.

Optionally, steps c and d are performed sequentially, or preferably, at the same time. Optionally, the oligonucleotide of step c is covalently labeled with a reporter that generates a detectable signal, for example, a fluorescent moiety or a “molecular beacon”, in which case step d is unnecessary. A detectable signal, or signal change that is correlated with the particles measured light scattering, distinct fluorescence signal, or the combination of light scatter and fluorescence indicates that immobilized target has hybridized to oligonucleotide probe and is thereby an authentication signal. In this example, combining particle light scattering and distinct fluorescence signal enables that very few unique target nucleic acid sequences need to be synthesized in order to achieve very high complexity codes. For example, selecting microparticle entities having two distinct light scatter signals, 2 colors of fluorescence, and 5 levels of fluorescence can be classified into 20 possible clusters. If either of two possible target nucleic acid sequences is used in conjunction with all possible combinations of these clusters, the number of possible codes is very large.

Optionally, the count or relative count of microparticles per cluster can be a component of the code, which results in extraordinarily large numbers of possible codes from small numbers of unique target nucleic acid sequences. Again, the methods of this embodiment provides for “lock & key” assurance, as above. Any particle analysis method or device capable of distinguishing particles from background and one target-specific particle from another by detecting particle-associated scattering and/or fluorescence, and which is capable of detecting target-correlated signal, can be used in the practice of this embodiment. Laser flow cytometric methods are preferred. Fluorescence microscopic methods and devices can also be employed. Laser scanning methods and devices also can be used, for example.

Any particle analysis method or device capable of distinguishing particles from background and one target-specific particle from another by detecting particle-associated scattering and/or fluorescence, and which is capable of detecting target-correlated signal, can be used in the practice of this invention. Laser flow cytometric methods are preferred. A laser flow cytometer useful in the practice of the present invention is the ORTHO CYTORONABSOLUTE® Flow Cytometer from Ortho-Clinical Diagnostics, Inc., Raritan, N.J. Fluorescence microscopic methods and devices can also be employed. Laser scanning methods and devices also can be used, for example, the Compucyte Laser Scanning Cytometer from Compucyte Corporation, Cambridge, Mass. Methods that permit spatial resolution of substrate-immobilized oligonucleotides can be used; laser scanning methods are particularly suitable. Methods and devices that are capable of distinguishing particles over background and resolving scattering or fluorescence signal from target-specific particles, and detecting target-correlated signal, as described above, that do not rely on physical separation of individual particles also can be employed.

In another aspect, the invention relates to product authentication using an authentication code comprising an amount of a plurality of distinct target nucleic acids, said method comprising:

A) forming a mixture, preferably in situ, comprising
i) immobilized the target nucleic acids, and
ii) target-specific particles, wherein a target-specific particle has immobilized thereto an oligonucleotide comprising a nucleic acid sequence complementary to at least a portion of one distinct target nucleic acid, wherein each particle independently is
   a) capable of scattering electromagnetic radiation of wavelength greater than or equal to about 200 nm, or

b) comprises a distinct first compound or plurality of compounds capable of producing a distinct fluorescence signal corresponding to the particle, or

c) capable of scattering electromagnetic radiation of wavelength greater than or equal to about 200 nm and comprises a distinct first compound or plurality of compounds capable of producing a distinct fluorescence signal corresponding to the distinct particle.

Additionally, the mixture, optionally, can be heated to denature duplex nucleic acid then cooled to allow hybridization of the target nucleic acid to the immobilized oligonucleotide or, if desired, the target nucleic acid can be heated prior to combination with immobilized oligonucleotide.

The method for determining multiple target nucleic acids, involves a step for producing a detectable signal, preferably an optical signal, most preferably fluorescence, correlated with target hybridized to immobilized oligonucleotide. A second compound capable of binding to duplex nucleic acid and producing a detectable fluorescence signal when bound thereto is combined with the mixture, or the oligonucleotide has linked thereto a second compound capable of fluorescence and the mixture is contacted with single-strand specific endonuclease, or the oligonucleotide or substrate comprises a second compound capable of fluorescence and the substrate or oligonucleotide comprises a fluorescence quenching compound in sufficient proximity to the second compound to quench fluorescence of the second compound prior to hybridization of target nucleic acid to immobilized oligonucleotide.

Using the above embodiments for producing a detectable signal correlated to target hybridized to immobilized probe, the mixture, in the case of particulate substrates, is exposed to, or introduced into a device or instrument, as described above, capable of detecting scattered electromagnetic radiation from a particle or upon detecting fluorescence from a first compound or plurality of compounds of a particle, or both, and detecting the fluorescence signal from the second compound as a measure of the presence of each distinct target nucleic acid. This can be accomplished because the target-specific particles can be distinguished by their specific light scattering and/or fluorescence. The second compound that provides target-correlated fluorescence signal, therefore, can be the same for each target. However, distinct second compounds for each target can be used if desired.

Fluorescence that may be associated with target nucleic acid and non-target nucleic acid free in solution, not hybridized to immobilized probe, does not contribute substantially, if at all, to the measured target-correlated signal.

In another aspect, the invention relates to a nucleic acid based product authentication kit wherein the product authentication code is encoded by a signature array of a population of particles associated with the product, wherein the signature array comprises information about the counts or relative counts of particles of at least two distinct clusters of particles within the population, comprising in the same or separate containers:

1) particles
a) capable of scattering electromagnetic radiation of wavelength greater than or equal to about 200 nm, or
b) comprising a compound or plurality of compounds capable of producing a fluorescence signal corresponding to the particle, or
c) capable of scattering electromagnetic radiation of wavelength greater than or equal to about 200 nm and comprising a compound or plurality of compounds capable of producing a fluorescence signal corresponding to the particle;
   2) an oligonucleotide comprising a nucleic acid sequence complementary to at least a portion of the target nucleic acid; and
   3) a compound which is capable of binding to duplex nucleic acid and which upon binding or being bound thereto is capable of producing a detectable signal.

All of the above methods or kits, can be configured for determining a single target nucleic acid or a plurality of target nucleic acids on a product to be authenticated. Further, the target nucleic acid and/or the microparticles comprising the authentication code may be contained in an authentication pouch associated with the product to be authenticated. As used herein, the term “immobilization” or “immobilized” means any means of spatially confining the subject nucleic acid, as may involve chemical, physical or physicochemical methods of immobilization.

### BRIEF DESCRIPTION OF THE DRAWINGS

FIG. 1 is a schematic diagram depicting the hybridization and detection of target nucleic acid to hybridized to particle-immobilized oligonucleotide.

FIG. 2A is a plot of forward-angle vs right-angle light scattering (particle size selection) thiazole orange as target DNA indicator, in presence of CTDNA, target DNA was not present.

FIG. 2B is a plot of forward-angle vs right-angle light scattering (particle size selection), thiazole orange as target DNA indicator, 1000 femtomoles of target DNA in an excess of CTDNA.

FIG. 2C is a plot of forward-angle light scattering vs green fluorescence, thiazole orange as target DNA indicator in the presence of CTDNA, target DNA not present.

FIG. 2D is a plot of forward-angle light scattering vs green fluorescence, thiazole orange as target DNA indicator, 1000 femtomoles of target DNA in an excess of CTDNA.

FIG. 2E is a plot of the distribution of events vs green fluorescence, thiazole orange as target DNA indicator in the presence of CTDNA, target DNA not present.

FIG. 2F is a plot of the distribution of events vs green fluorescence, 1000 femtomoles of target DNA in an excess of CTDNA.

FIG. 3 is a plot of the mean green fluorescence vs copy number of double-strand target DNA.

FIG. 4 shows a schematic of nuclease protection and detection of particle-associated target nucleic acid.

FIG. 5 is a plot of mean channel orange fluorescence (stained with streptavidin-conjugated R-phycoerythrin, nuclease protected dsDNA captured on beads) vs copy number of double-strand target DNA.

FIG. 6 is a plot of mean channel fluorescence vs PCR amplification cycle number.

### DETAILED DESCRIPTION OF PREFERRED EMBODIMENTS OF THE INVENTION

All publications cited below are hereby incorporated by reference. Unless defined otherwise, all technical and scientific terms used herein will have the commonly understood meaning to one of ordinary skill in the art to which this invention pertains.

It must be noted that as used herein and in the appended claims, the singular forms “a,” “an,” and “the” include plural reference unless the context clearly dictates otherwise. Thus, for example, a reference to “a population of entities” is a reference to one or more populations of entities and includes equivalents thereof known to those skilled in the art and so forth.

As used herein, the term “array” means a collection of data items arranged in such a way so that each data item in the array can be located.

As used herein, a “cluster of entities” or a “cluster” means a classification of at least two entities that are grouped together because they share one or more discretely measurable common properties. In particular embodiments of the invention, the entities within “a cluster of entities” share one, two, three, four, five, six, seven, eight, nine, ten, or more discretely measurable common properties.

As used herein, the “count of entities per cluster”, the “number of entities per cluster”, the “count (or number) of entities within a cluster”, and the “count (or number) of entities of a cluster” are used interchangeably to mean the number or sum total of entities within a cluster. The “count of entities per cluster” can be obtained by counting discrete entities within the cluster by means such as an automated counter or manual counting method.

As used herein, a “discretely measurable common property” is a property of or associated with each individual entity within a single cluster, and said property can be measured from the individual entity. The discretely measurable common property allows an entity to be assigned into a particular cluster. Entities having the same set of one or more discretely measurable common properties can be assigned into the same cluster. Entities having different sets of discretely measurable common properties can be assigned into distinct clusters.

Examples of “discretely measurable common property” include, but are not limited to, the properties of one or more tags associated with entities of a cluster, such as the fluorescent intensity or spectra when the entity is labeled with a fluorescent tag, the sizes of the entities, the shape of the entities, and other properties of the entities, such as being magnetic or not, density, or solid characterization, or the nucleotide sequence or amino acid sequence when the entities are composed of nucleic acid molecules or peptides/polypeptides.

As used herein, “distinct clusters of entities” means clusters that are different because entities within one cluster having at least one discretely measurable common property that is not shared with the entities within the other cluster(s). Thus clusters of entities can be distinguished from one and another by the measurement of any of the discretely measurable common properties shared by entities within one cluster but not by entities within the other cluster(s)—the distinct discretely measurable common properties.

For example, the clusters of entities can be distinguished by sizes, density or solidity including elasticity, brittle fracture, water-content etc. The particle size can be measured, for example, in a flow cytometry apparatus by so-called forward or small-angle scatter light or by microscopic examination. The clusters of entities can also be distinguished by shape. The shape of the particle can be discriminated, for example, by flow cytometry, by high-resolution slit-scanning method or by microscopic examination. The shape of a printed dot, for example, can be measured by a scanner. The clusters of entities can further be distinguished by tags, such as by fluorescent dyes with different emission wavelengths. Even when they are labeled with the same tag(s), the clusters of entities can still be distinguished because of different concentrations, intensity, or amounts of the tag associated with the entities, or the different ratios of tags on individual entities. Clusters of entities can be distinguished even when all entities share one or more discretely measurable common properties (e.g., particle size and particle shape), but do not share at least one other discretely measurable common property (e.g., intensity or amount of fluorescent tag per entity).

Methods known to a person skilled in the art can be used to measure the quality or quantity of tags. In addition, the clusters of entities can be differentiated by other property or characteristic of the entities, such as being magnetic or not. When the entities are composed of or labeled with nucleic acid or peptide molecules, the clusters of entities can be differentiated by their sequences.

As used herein, the term “entity” means a thing or composition that can exist separately or independently from other things. Examples of entities that can be used in the present invention include, but are not limited to, microparticles, nucleic acid molecules, or peptides/polypeptides.

As used herein, the terms “microparticle”, “microsphere”, “microbead”, “bead”, “microsphere”, and “particle” are used interchangeably and bear equivalent meanings with respect to their particulate nature, understanding that particles can have various shapes and sizes. Preferred particles range in size from approximately 10 nm to about 200 μm in diameter or width and height in the case of nonspherical particles. For example, the particles can have a size of 0.05-50 μm, 0.1-20 μm, 1-20 μm, or 3-10 μm in diameter. The microparticles can have a different shape, such as a sphere, cube, rod or pyramid.

Those of ordinary skill in the art can use microspheres of various compositions. For example, styrene monomers polymerized into hard rigid latex spheres have been used as calibration aids at high magnifications. These latex spheres are known for their high level of inertness in the electron beam, and clusters constructed from groups of such particles within non-overlapping size ranges of approximately 0.05 to 2 microns may be detected by electron microscopy or light-scattering investigations. Likewise, the particles can be made of many other types of materials. For example, the microparticles can be made of polystyrene or latex material. Other types of acceptable polymeric microspheres include, but are not limited to, brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, POLYOX, EUDRAGIT, sugar spheres, hydrofuran, PLGA (poly(lactic coglycolic acid)) or combinations thereof. In general, such particles can be made by a copolymerization process wherein monomers, e.g., unsaturated aldehydes or acrylates, are allowed to polymerize in the presence of one or more tags, e.g., fluorescein isothiocynate (FITC), in the reaction mixture (see for example U.S. Pat. Nos. 4,267,234 issued to Rembaum; 4,267,235 Rembaum et al; 4,552,812, Margel et al.; 4,677,138, Margel). The microparticles can be produced, for example, by extrusion or spherenization. Rolland, et al., J. AM. CHEM. SOC. 9 VOL. 127, NO. 28, 2005 describe the production of exemplary uniform, sub-micron, biodegradable particles produced in different shapes, such as a spheres, cubes, rods or pyramids, that can optionally encapsulate fluorescent material, among other things.

To increase the per volume information content, the entity can be labeled with one or more tags that are visible or invisible to naked eyes. The term “tag” or “taggant” as used herein can be any composition that is suitable for the purpose of detecting or identification. The tag can be overt, covert, or invisible or otherwise difficult to detect on individual entities or small numbers of entities, yet having an overt signal detectable from all or a larger number of entities. For example, the entity can be labeled with one or more colors, fluorescent dyes, ultraviolet radiation dyes, luminescent compositions, hapten, nucleotides, polypeptides, or scents. A single entity can be labeled with more than one tag of the same or different types. For example, a particle can be labeled with two or more discretely distinguishable dyes in varying proportion; or a particle can be labeled with a nucleotide and a fluorescent dye. Any of the known tags and the combinations of the tags with entities can be used in the invention. Methods known to those skilled in the art can be used to label an entity with one or more tag. For example, U.S. Pat. No. 6,632,526 teaches methods of dyeing or staining microspheres with at least two fluorescent dyes in such a manner that intra-sample variation of dye concentrations are substantially minimized. The entity can be a segmented particle whose composition is varied along the diameter or the length of the particle. U.S. Pat. No. 6,919,009 teaches methods of manufacture of rod-shaped particles. In one particular embodiment, the entity can be an entity that is labeled with or affixed to other entities. For example, the entity can be a symbol printed with an ink containing microparticles. Another example of an entity, according to this embodiment, is a particle that is covalently or non-covalently affixed with one or more other particles. US Patent Application Publication No. 2006/0054506 describes submicron-sized particles or labels that can be covalently or non-covalently affixed to entities of interest for the purpose of quantification, location, identification, tracking, and diagnosis.

The entity that can be used in the present invention preferably can be ingestible and/or non-toxic in amounts used. For example, the entity can be a liposome microparticle, i.e., a particle formed by a lipid bilayer enclosing an aqueous compartment. The entity can also be a microparticle made of pulverized cellulose material, see for example the abstract of JP0 6,298,650. The entity can further be microparticles made of calcium, such as milk calcium, inorganic calcium or organic calcium. For example, edible oil-containing calcium microparticles can be obtained following the teaching of U.S. Pat. No. 6,159,504. Biodegradable polymers, such as dextran and polylactic acid, can also be used to prepare ingestible microparticles. In addition, the edible microparticles include solid lipophilic microparticles comprising a lipophilic substance, hyaluronic acid or an inorganic salt thereof. Exemplary lipophilic particles are disclosed in US Patent Application Publication No. 20030064105.

The entity can be magnetic. U.S. Pat. No. 6,773,812 describes hybrid microspheres constructed using fluorescent or luminescent microspheres and magnetic nanoparticles. Distinct clusters of microspheres can be constructed based on fluorescent intensities by analogy to the clusters described in Example 1 infra, and as provided in International Patent Application Publication No. WO 01/464774, and separations can be affected based on the variable degree of magnetic content to aid in the analysis of the cluster membership on devices like the Immunicon CELLSEARCH instrument. The various microspheres disclosed in U.S. Pat. No. 6,773,812 can be used in the present invention. The particles can also have any other property that facilitates collection, separation, or identification of the particles.

The entity can also be made of chemically inert materials to enhance the survival of the entity in a chemical or biological environment, including materials resistant to heat, high or low pH, etc. The entity can further be made of materials that are non-toxic, or materials that can serve as carriers for the active ingredient. The entity can even be made from the active ingredient of a pharmaceutical product.

As used herein, a “population of entities” or a “population” means a collection of a combination or plurality of entities that include two or more distinct clusters of entities, wherein entities within one cluster have one or more discretely measurable common properties that are different from that of entities within another cluster from the same population.

As used herein, the term “relative counts of entities per cluster” means a ratio of the count of entities per cluster relative to another number. In some embodiments, the other number is the count of entities within a different cluster. In other embodiments, the other number is the total count of entities within two or more clusters of a population of entities. In other embodiments, the other number is representative of the amount or concentration of the cluster or the population of entities, such as unit volume or weight of the cluster or the population of entities. In yet other embodiments, the other number is representative of the amount or concentration of a product the cluster is associated with, or the amount or concentration of a portion or a component of the product.

As used herein, the term “a representative number of entities within a population of entities” refers to a fraction or a portion of the population of entities which contains the same clusters of entities and the same count of entities per unit of each cluster as those of the population.

For illustrative purpose, in one specific embodiment of the invention, the population of entities is composed of microparticles each simultaneously labeled with two or more fluorescent dyes, for example, according to U.S. Pat. No. 6,632,526 or U.S. Pat. No. 6,649,414. The microparticles can also be purchased from a commercial source, such as Luminex Corporation (Austin, Tex.). For example, the particles can be labeled with two dyes, such as a red fluorescent dye, 1,3-bis[(1,3-dihydro-1,3,3-trimethyl-2H-indol-2-ylidene)methyl]-2,4-dihydroxy-cyclobutenediylium, bis(inner salt) (Dye 1) and an orange fluorescent dye, 2-(3,5-dimethylpyrrol-2-yl)-4-(3,5-dimethyl-2H-pyrrol-2-ylidene)-3-hydroxy-2-cyclobuten-1-one (Dye 2). As is readily appreciated, other combinations of dyes with other colors and other chemical compositions can also be used to label microparticles. One skilled in the art can select among a variety of suitable dyes such as, for example, the dyes recited in U.S. Pat. No. 6,649,414, depending upon desired emission/absorption and hydrophobic properties, etc. Where fluorescent dyes are used, the dyes are chosen such that the emission maxima of the dyes used preferably falls about in the center of the fluorescence detection channels of the measurement device used. Preferably the dyes used have emission maxima separated by greater than 10 nm, 25 nm, or 50 nm from each other.

Microparticles within the population are heterogeneous because they do not share at least one distinctly measurable property (e.g., intensity or amount of fluorescent tag per entity). The fluorescent intensity of the red or orange dye on each microparticle can be measured by flow cytometry.

As used herein, “a signature array of a population of entities” is an array comprising information about the counts or relative counts of entities of at least two distinct clusters of entities within the population. For example, a signature array comprises information about the counts of microparticles within each distinct cluster of the population. Each cluster is different from one another by at least one distinct discretely measurable common property.

The method of product authentication of the present invention uses a product authentication code defined by the nucleotide sequence of a target nucleic acid and/or signature array of a population of entities, which has high per volume information content.

As used herein, a “product authentication code” or “product identification code” is a system that represents information specific to a product. The system or code is matched with a particular product or batch of products such that tacking or sampling of the code associated with the particular product or batch of products provides those individuals designated by the source originator of the code or the commercial user of the code to know any of a variety of characteristics or information about the product(s). For example, a “product authentication code” for a pharmaceutical product can represent information about the product, such as the chemical composition, the concentrations of the effective ingredients, the date or place of manufacture, the source of distribution, the batch, the shelf life, or a myriad of other information designations.

For the purposes of the present invention, the term “substrate” represents any particulate or non-particulate material to which or upon which an oligonucleotide can be immobilized.

In certain embodiments, nucleic-acid based product authentication method of the present invention for detecting a target nucleic acid rely on a set of measurements of a first parameter or parameters in order to identify an entity within a population of entities comprising a signature array. A corresponding set of measurements of a second parameter or parameters is made to determine the presence of, or to quantify a target nucleic acid. The entity identification and target determination parameters are used to generate a correlated data list. These parameter measurements can be made sequentially. Each set of measurements is referred to as an event. Thus, a data list can appear as follows:

| Event | Parameter X | Parameter Y | parameter Z |
|---|---|---|---|
| 1 | X1 | Y1 | Z1 |
| 2 | X2 | Y2 | Z2 |
| 3 | X3 | Y3 | Z3 |
| — | — | — | — |
| n | Xn | Yn | Zn |

Such data lists are typically obtained using laser scanning cytometry or flow microfluorimetry. They are generally referred to as “list-mode data.”

A range of values for a single parameter X (“A”) can be used to define a cluster of entities that bear a first target nucleic acid. Another range of values for X (“B”) can be used to define for a cluster of entities that bear a second target nucleic acid. By this means, both assays for both the first and second target nucleic acid can be carried out simultaneously. Whenever an event is correlated with a value for X, in range A, the measurement of parameter Z would be for the first target nucleic acid. Whenever an event is correlated with a value for X, in range B, the measurement of parameter Z would be for the second target nucleic acid. The number of assays for different target nucleic acid that can be carried out simultaneously is limited only by the number of discrete ranges that can be detected within the parameter X being used to specify an to entity for each target nucleic acid. A high degree of certainty can be obtained that events belong to a particular entity class (i.e., cluster) by setting broad ranges for A, B, etc.

The number of assays that can be carried out simultaneously can be increased if more than one parameter is used to define a cluster of entities that bear a target nucleic acid. A range of values for parameter X (“A”) in conjunction with a range of values for parameter Y (“Q”) can be used to define a cluster of entities that bear for a first target nucleic acid. Another range of values for X (“B”) in conjunction with another range of values for parameter Y (“R”) can be used to define a cluster of entities that bear for a second target nucleic acid.

By this means, assays for both the first and second target nucleic acid can be carried out simultaneously. Whenever an event is correlated with a value for X, in range A, and Y, in range Q, the measurement of parameter Z would be for the first target nucleic acid. Similarly, whenever an event is correlated with a value for X, in range B, and Y, in range R, the measurement of parameter Z would be for the second target nucleic acid. When using multiple parameters to identify each cluster of entities that bear a target nucleic acid, the number of assays for different target nucleic acid/cluster combinations that can be carried out simultaneously is no longer limited by the number of discrete ranges of an individual parameter, since a specific combination of parameters can be used to specify an assay for each target nucleic acid. Additionally, a higher degree of certainty can be obtained that events belong to a particular assay class by setting broad ranges for A, B, Q, R, and so on.

In laser scanning cytometry or flow cytometry, the preferred parameters for classifying multiple simultaneous assays are forward light scatter, side scatter, or a fluorescence parameter(s) distinct from that used to detect signal from hybridized nucleic acids. For DNA chips, the preferred parameters for classifying multiple assays are spatial dimensions, x and y coordinates or positions on the surface of the chip.

A major advantage of the present invention, in addition to allowing multiple assays to be carried out simultaneously, is the ability to obtain replicate measurements of the same assay using the list mode data. For example, events 1 through n are classified as belonging to an assay for a first target nucleic acid, based upon measurements within a range of values for parameter X (“A”) in conjunction with measurements within a range of values for parameter Y (“Q”). Events n+1 through p are classified as belonging to an assay for a second target nucleic acid, based upon measurements within a range of values for X (“B”) in conjunction with measurements within a range of values for parameter Y (“R”).

Statistical analysis can be performed to determine the mean and standard deviation of Z for the first assay by analyzing events 1 through n. Similarly statistical analysis can be performed to determine the mean and standard deviation of Z for the second assay by analyzing events n+1 through p. Thus, differences in signal between a target and control and between target levels can be expressed as differences between means of replicate measurements, thereby, enhancing the sensitivity of all assays performed. Events belonging to a particular assay class do not have to be measured sequentially. They have been shown as occurring sequentially for illustrative purposes only.

Another advantage is that a single parameter can be used to detect the signal from multiple target nucleic acids simultaneously. Thus, only one reporter element is necessary, regardless of the number of target nucleic acids to be determined. Conjugates of fluorescein isothiocyanate (FITC) are useful for signal generation. In preferred embodiments, an intercalating dye is used.

The determination of the presence or amount of double-strand or duplex nucleic acid in the presence of single-strand nucleic acid can be accomplished using a compound which upon binding or when bound to duplex nucleic acid, produces a detectable change in an optical property such as absorption or fluorescence (Ririe et al., Anal Biochem 245, 154 (1997), Wittwer et al., BioTechniques 22, 130 (1997), Yamamoto et al., European Patent Publication 0 643 140 A1 and U.S. Pat. Nos. 5,049,490 and 5,563,037 to Sutherland et al.).

Nucleic acids can be determined using a “nuclease protection assay” as described in Sambrook et al. in Molecular Cloning: A Laboratory Manual, Vols. 1-3, 2nd ed., Cold Spring Harbor Laboratory Press, Cold Spring Harbor, N.Y. (1989) and Thompson et al., Biol. Chem. 267, 5921 (1991). This method involves hybridization of a labeled, single-strand DNA probe to a target DNA or RNA molecule and subsequent hydrolysis of single-strand nucleic acid by a single-strand specific endonuclease, such as S1 nuclease. Hybridized duplex nucleic acid remains intact, protecting the labeled probe from hydrolysis by the endonuclease. The label can be a dye, a fluor, a radiolabeled molecule, an enzyme, and so on as recognized in the art, and appropriately detected. The assay can be quantitative for the target nucleic acid.

Nucleic acid amplification methods, such as the Polymerase Chain Reaction (PCR), often provide for the detection of a target nucleic acid using a labeled probe or, alternatively, a labeled primer that is extended into detectable product captured onto an immobilized complementary oligonucleotide probe. A wide variety of labels have been developed, Vlieger et al., Anal Biochem 205, 1 (1992), Yang et al., Blood 81, 1083 (1993). Fluoresceinated primers have been used in flow cytometric detection of bcl-2/MBR and IgH gene rearrangements, Barker et al., Blood 83, 1079 (1994).

Another method for determining a target nucleic acid involves linear amplification of signal from a target-specific oligonucleotide probe labeled with a fluorophor. The labeled probe, when hybridized to target, is hydrolyzed by a duplex nucleic acid-specific exonuclease, such as exonuclease III, which hydrolyzes duplex DNA (dsDNA) from the 3′ terminus. Truncated hetero-duplex hybrids produced during hydrolysis are unstable. Shortened fragments of labeled probe dissociate. Fresh, intact, labeled probe can then hybridize to the target and a new round of hydrolysis occurs followed by dissociation of shortened, labeled probe and so on. The resulting probe fragments are then separated by electrophoresis and determined using a sequencing apparatus. (Okano et al. Anal Biochem 228, 101 (1995)).

Tyagi et al., Nature Biotechnolgy 14, 303 (1996), have described the use of, so-called, molecular beacons for the detection of nucleic acids. A molecular beacon is a target-specific oligonucleotide comprising a fluor, and in close proximity to the fluor, a quencher. Upon binding of target nucleic acid, the fluorophore and quencher become separated, and the resulting fluorescence is detected.

In the case of a plurality of distinct target nucleic acids, a target-specific substrate/particle is rendered specific to a distinct target nucleic acid by having immobilized thereto an oligonucleotide complementary to at least a portion of only one distinct target nucleic acid. Target DNA can be in single-strand (ssDNA) or duplex form. If in duplex form, it can be treated, usually by heating, to denature the dsDNA prior to, during, or subsequent to combination with a substrate-immobilized oligonucleotide. The ssDNA target is then allowed to hybridize to substrate-immobilized oligonucleotide.

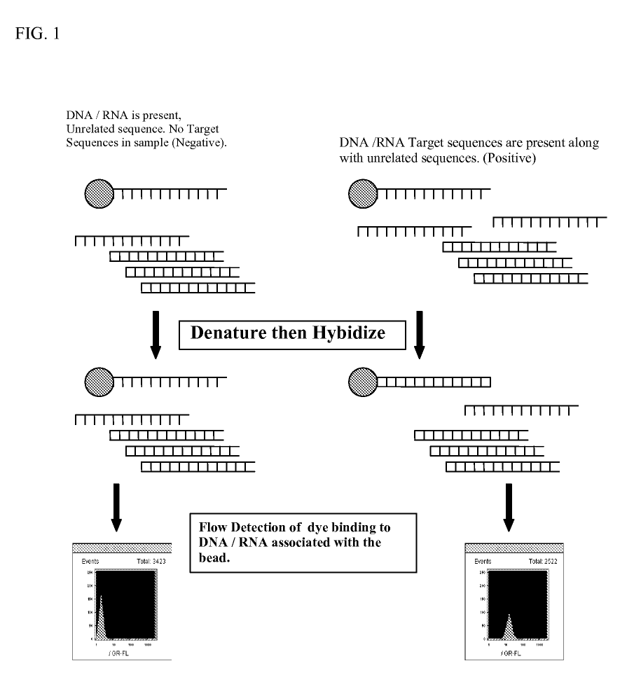

Any optical signal and method of producing it that is derivable from or associated with target nucleic acid hybridized to substrate-immobilized oligonucleotide can be used in the practice of this invention. As noted, in one embodiment, target-correlated signal is obtained by utilizing a dyefluorophore that binds to dsDNA and produces a detectable fluorescence signal when so bound. This is illustrated schematically in FIG. 1. Alternatively, a fluorescence signal associated with target hybridized to substrate-immobilized oligonucleotide can be produced by utilizing a fluorophore linked to the oligonucleotide. When the substrate-immobilized oligonucleotide probe is hybridized to target nucleic acid to form duplex nucleic acid, single-strand specific endonuclease is not able to hydrolyze or substantially hydrolyze the oligonucleotide, or therefore, the portion of oligonucleotide having the fluorophore linked thereto. Thus, fluorophore linked to oligonucleotide remains associated with the substrate; target-correlated signal originating from the fluorophore that is associated with hybridized target is detected. Signal originating from fluorophore that is released by single-strand endonuclease mediated hydrolysis does not substantially contribute to the measured fluorescence.

In a related embodiment, restriction endonuclease mediated hydrolysis can be used to release a fluorophore bound to the oligonucleotide, upon creation of a double strand DNA sequence that contains a restriction site when target hybridizes to substrate-immobilized oligonucleotide. In another embodiment, a fluorophore is bound to the oligonucleotide or the substrate so as to be sufficiently close to a compound capable of substantially quenching (by greater than or equal to about 50%) its fluorescence. If the fluorophore is linked to the substrate then the quencher is linked to the oligonucleotide. If the quencher is linked to the substrate then the fluorophore is linked to the oligonucleotide. Or fluorophore and quencher can both be linked to the oligonucleotide.

The ability of a dye to produce a detectable signal only when bound to dsDNA, although preferred, is not a requirement of a dye in order for it to be useful in practicing the invention.

### Particles and Particle-Immobilized Oligonucleotide

Particle substrates of the present invention may be composed of any material or combination of materials that will enable covalent or non-covalent linking of oligonucleotide and/or other compounds such as fluorophores. They can be of a construction that allows compounds such as fluorophores to be encapsulated, as long as the fluorescence is detectable. They can be of a size and composition so as to scatter electromagnetic radiation. Preferably, the particles are composed of one or more polymers comprising ligands or functional groups that enable non-covalent or covalent bonding of oligonucleotides and/or other compounds directly or through suitable binding partners or linker groups. Preferably, they are substantially spherical and have a diameter in a range from about 0.3 microns to about 50 microns, more preferably 0.9 microns to about 15 microns and/or are capable of scattering electromagnetic radiation greater than or equal to about 200 nanometers. Polymeric particles for use in the practice of the invention can be prepared by methods known to the skilled artisan. See, for example, U.S. Pat. Nos. 4,997,772, 5,149,737, 5,210,289 and 5,278,267 and references cited therein. Alternatively, suitable particles can be obtained for instance, from Bangs Labs, Fishers, Ind. and Spherotech, Libertyville, Ill. and others known to the skilled artisan. The particles may be attached in or on to the articles to be authenticated through various means known in the art. Particle retention can be achieved using appropriate materials, for example, a mesh incorporated into the product or binding agents such as starches or sprays having adhesive properties.

The attachment of oligonucleotides to particulate and non-particulate substrates can be carried out using methods that are well known, as described, for example, in U.S. Pat. Nos. 5,177,023, 4,713,326, 5,147,777, 5,149,737, and EP-B-0 070 687, and International Patent Application Publication No. WO-A-88/01302, and references cited therein. An oligonucleotide can have linked to it any desired compound or compounds as long as the compound or compounds do not substantially interfere with hybridization of the target nucleic acid. For example, a ligand such as biotin, a fluor, a fluorescence quenching compound, or other compound(s) can be so linked. An oligonucleotide can be linked to a particle at its 3′ or 5′ end.

An oligonucleotide probe can comprise any desired number of bases. In general, it can comprise a base length between about 5 to about 10⁵ nucleotide bases. Preferably, it comprises a base length between about 15 to about 40,000 bases, more preferably between about 30 to about 10,000 bases, and even more preferably between about 30 to about 1000 bases, and most preferably between about 30 to about 500 bases.

### Target Nucleic Acid Spotting

One method of immobilizing target nucleic acid unto products to be authenticated is to use the “sticky” polymer method of Sheu, Jue-Jei; et al., US Patent Application Publication No. 2005/0008762, the disclosure of which is incorporated herein by reference. In the Sheu, Jue-Jei method, a water-insoluble medium comprising polymeric substances is first dissolved in an organic solvent to form a medium/solvent mixture. Then, target nucleic acid solution in water, TE buffer or other suitable buffer, is mixed with an intermediate solution, such as methanol, ethanol, acetone, glycerol or their mixtures to form a homogenous mixture of nucleic acid solution. The two mixtures are mixed to form a third homogenous mixture which is then spotted or spread on the product to be authenticated. After drying, the nucleic acid taggants protected by the water-insoluble medium adhere on the surface of the object. A liquid object may be similarly marked.

The water-insoluble medium comprises polymeric substances such as polypropylene (PP), polymethyl methacrylate (PMMA), polycarbonate (PC) and polystyrene (PS). The organic solvent could be any one of chloroform, dichloromethane and benzole solvent, such as xylene or toluene or other organic solvent known in the art.

The gene pen apparatus of Friedman, et al., U.S. Pat. No. 6,235,473, may be adapted for printing target oligonucleotides unto products to be authenticated. Friedman et al.'s apparatus, the entire disclosure of which is incorporated herein by reference, comprises a reservoir containing an oligonucleotide, the reservoir being fluidly connected to a printing head, the printing head comprising a flow controlling means, especially a pin valve means or a felt tip means for printing target nucleic acids on a product.

### Detection

An important aspect of the invention with particulate substrates is the use of methods and instrumentation capable of distinguishing particles and target-correlated signal over background, and target-specific particles, one from another, based on the light scattering and/or fluorescence properties of the particles. Target-specific particles are distinguishable, one from another, by the distinct differences in their light scattering properties and/or the distinct differences in the fluorescence signals derived from a fluorophore or plurality of fluorophores associated with each target-specific particle, which fluorescence signals are distinct from target-correlated fluorescence.

Light scattering by a particle depends on its size and/or refractive index, both of which can be modified as desired using well known methods. It is not necessary for a particle to be capable of scattering light. Particle discrimination can be achieved by incorporating, encapsulating, non-covalently or covalently bonding to a particle one or more compounds that are capable of producing distinct fluorescence.

Laser scanning cytometry has been used for distinguishing particles, such as blood cells. When a cell or group of cells (agglutinate) is scanned by the laser light beam, the illuminating light is scattered by the cell or group of cells; the intensity of scatter being a function of cell (or agglutinate) size and shape. For example, individual red blood cells scatter less light than small agglutinates, which in turn scatter less light than large agglutinates.

Similarly, when a cell or group of cells (agglutinate) is scanned by the laser light beam, the illuminating light can induce fluorescence from a fluor(s) associated with a cell or cells. If a fluorophore is relatively uniformly associated with a cell, the fluorescence intensity is related to agglutinate size. For example, individual red blood cells would fluoresce less than small agglutinates which in turn, would fluoresce less than large agglutinates.

Using scattered light and fluorescence in combination is more reliable than using either alone for discriminating different classes of agglutinates.

In flow cytometry, particles, such as blood cells, are introduced into the center of a fast moving fluid stream and forced to flow single file out a small diameter orifice at uniform speeds. The particles are hydrodynamically focused to the center of the stream by a surrounding layer of sheath fluid. The particles within the stream pass a measurement station where they are illuminated by a light source and measurements, in the case of red blood cells, are made at rates of 2.5×10² to 10⁶ cells per minute. Laser light sources are used in the measurement of particles; typical laser light sources used include argon ion lasers (UV, blue and green light), krypton lasers (yellow and red light), helium-cadmium lasers (UV and blue light), and helium-neon lasers (red light).

In fluorescence microscopy, particles can be detected on a microscope slide or equivalent. Typically, they are illuminated by a white light source or a substantially monochromatic light source. Here too, laser light sources may be used as the source of the monochromatic light. The presence of particles may be assessed with the white light, and the associated fluorescence assessed with monochromatic light and appropriate filters. Visual or automated means may be used for one or both of these readings.

A preferred flow cytometer is capable of selecting for the detection of target-correlated signal associated with particles having a defined range of forward-angle and right-angle scattering signal intensity or particular fluorescence (Yang, et al. Blood 81, 1083 (1993), Barker et al. Blood 83, 1079-1085 (1994), Chandler, et al., ISAC XIX International Congress, Colorado Springs, Colo. USA, Fulton, et al., Clinical Chem 43, 1749 (1997), Fulwyler, et al., Methods Cell Biology, Second Edition, Academic Press, v 33, 613 (1990), and McHugh, Methods Cell Biology, Second Edition, Academic Press, v 42, 575, (1994)). Data acquisition is initiated by light scattering and/or fluorescence associated with a particle. Selecting for signal associated with a particle enables the detection of target-correlated signal without interference from fluorescence originating from the bulk solution phase in which the particles are immersed. Thus, the signal/noise ratio is large. Target-correlated signal is proportional to the amount of target, and determination of multiple target nucleic acids is also possible using the preferred flow cytometric methods. Multiplex analysis of nucleic acids that are free in solution using flow cytometry has been described by Chandler et al., ISAC XIX International Congress; Colorado Springs, Colo. (1998), Fulton et al., Clin Chem 43 1749 (1997), Fulwyler et al., Methods Cell Biology, 2^{nd} Ed. 613 (1990), and McHugh, Methods Cell Biology, 2^{nd} Ed. 575 (1990).

### DNA Binding Fluorophores

Numerous compounds capable of binding to dsDNA and producing a detectable signal when bound thereto or which can be chemically modified to produce a detectable signal are known and available to the skilled artisan. Included among them are dyes, antibiotics, and chemotherapeutic agents. They can be intercalating or non-intercalating; but, they must not bind substantially to the substrate to which oligonucleotide is immobilized. Specific examples include, but are not limited to, acridine orange, propidium iodide, ethidium bromide, mithramycin, chromomycin, olivomycin, see also, U.S. Pat. Nos. 5,049,490 and 5,563,037. Preferred compounds include, Hoechst H33258, Hoechst H33342, DAPI (4′,6-diamidino-2-phenylindole), and from Molecular Probes, TOPRO, TOTO, YOPRO, YOYO, SYBR GREEN I, Picogreen dsDNA Quantitation Reagent, and Thiazole Orange. Some compounds, such as YOYO, are virtually non-fluorescent until they bind dsDNA. Acridine orange has metachromatic properties that allow distinction between binding to ssDNA or dsDNA. For the purposes of the present invention, a compound either must not substantially bind to single strand immobilized oligonucleotide, but if it does, any resulting signal must be capable of being differentiated from that produced when the compound is bound to dsDNA.

### Single-Strand Specific Endonucleases, Restriction Endonucleases

Single-strand specific endonucleases that can be used in the practice of this invention include, but are not limited to, S1 endonuclease and mung bean endonuclease. Restriction endonucleases that can be used include, but are not limited to: Acc65 I, Acc I, Aci I, Alu I, Apa I, ApaL, Ava I, Ava II, Bae I, BamH I, Bcg I, Rd I, Bfa I, Bgl I, Bgl II, Bsa I, BsaJ I, Bsl I, BspH I, BsrG I, Bst4C I, BssS I, BstE II, BstU I, BstX I, BstY I, Cla I, Dde I, Dpn I, Dpn II, Dra I, Dra III, EcoO109 I, EcoR I, EcoR V, Fau I, Fok I, Hae II, Hae III, Hha I, Hinc II, Hind III, Hinf I, Hpa I, Hpa II, Kpn I, Mbo I, Mlu I, Mnl I, Mse I, Msp I, Nae I, Nco I, Nde I, Nhe I, Nla III, Nru I, Pst I, Pvu I, Pvu II, Rsa I, Sac I, Sac II, Sal I, Sau3A I, Sca I, Sfi I, Sma I, SnaB I, Spe I, Sph I, Ssp I, Stu I, Sty I, Taq I, Xba I, Xcm I, Xho I, Xma I, and Xmn I.

### Fluorophore and Quencher

In an embodiment wherein a fluorophore and a quencher are provided together, a fluorophore can be linked at or near a terminus of an oligonucleotide and a quencher can be linked at or near the other terminus, as in a stem-loop structure (molecular beacon) described by Tyagi et al., Nature Biotechnolgy 14, 303 (1996), Tyagi et al., Nature Biotechnolgy 16, 49 (1998), Giesendorf et al., Clin Chem 44, 482 (1998), and Estrada et al., Mol Cell Probes 12, 219 (1998). In a stem-loop structure the oligonucleotide comprises complementary nucleotide base pairs at both termini. Hybridization of the complementary base pairs results in formation of a closed loop with the fluorophore and quencher in sufficient proximity so that fluorescence is quenched. Upon hybridization of target nucleic acid to the oligonucleotide, the loop is opened and fluorophore and quencher become sufficiently separated to allow fluorescence.

In all cases, separation of substrate-immobilized oligonucleotide from the matrix in which it is immersed is not required prior to detection. However, such separation may be carried out, if desired, using well-known methods such as filtration, centrifugation or magnetic separation.

While this disclosure describes detection of PCR products by mixing these with microspheres and intercalating dyes, further disclosed is the addition of the microspheres-immobilized oligonucleotide probes and the intercalating dyes to the PCR reaction before, as an alternative to after, thermocycling. In this embodiment of the invention, either the dye (in this embodiment, preferably a thermostable dye) or the microspheres, alone, could be in the mix prior to thermocycling, or alternatively, both may be present. Accordingly, the amount of target oligonucleotide used and sample handling can be even further reduced when measuring PCR products either in the homogeneous formats described herein above and in Example 6 hereof, or in other formats that might be used or developed by one skilled in the art.

### MATERIALS AND METHODS FOR EXAMPLES

Unless indicated otherwise, the particles used in the examples were copolymers of (poly[styrene-co(p-vinylbenzylthio)proprionic acid] 97.6:2.4 molar ratio) prepared as described in U.S. Pat. Nos. 5,149,737; 5,210,289 and 5,278,267. The particles were substantially spherical and approximately 1.7 micrometers in diameter. Coupling of oligonucleotide to the particles was carried out essentially as described in U.S. Pat. No. 5,147,777. Unless indicated otherwise, oligonucleotides were synthesized using procedures well known in the art. Two oligonucleotides used in the examples are identified below:

SEQ ID NO: 1:
5′-TTTCCAAGTA AGCAATAACG TCAGCTCTTT CTTGTGGCTT
CTTCATACCA GCGAAAGACA TCTTAGTACC TGGCATGAAC
TTCTTTGGGT-3′.

The above oligonucleotide was modified by linking biotin to the 5′ terminus through two tetraethylene glycol (TEG-TEG) spacers with and without an aminodiol (ADL) linker at the 3′ terminus for attachment to a particle as represented by the following:

Oligo-1A:
Biotin-TEG-TEG-5′-TTTCCAAGTA AGCAATAACG TCAGCTCTTT CTTGTGGCTT
CTTCATACCAG CGAAAGACAT CTTAGTACCT GGCATGAACT TCTTTGGGT (SEQ ID
NO: 1)-3′-TEG-TEG-ADL-Particle.
and
Oligo-1B:
Biotin-TEG-TEG-5′-TTTCCAAGTA AGCAATAACG TCAGCTCTTT CTTGTGGCTT
CTTCATACCAG CGAAAGACAT CTTAGTACCT GGCATGAACT TCTTTGGGT (SEQ ID
NO: 1)-3′.
SEQ ID NO: 2:
5′-ACCCAAAGAA GTTCATGCCA GGTACTAAGA TGTCTTTCGC TGGTATGAAG
AAGCCACAAG AAAGAGCTGA CGTTATTGCT TTGGAAA-3′.

### Hybridization Conditions

Hybridization of biotinylated oligonucleotide probe, (SEQ ID NO:1) to target DNA (SEQ ID NO:2) was carried out by incubating 100 fmoles of probe in 10 μL of 0.15M potassium chloride, 0.01M tris(hydroxymethyl)aminomethane (Tris), 1 mM ethylenediaminetetraacetic acid (EDTA), pH 8.3 in the presence of 1 μg of calf thymus DNA. DNA was denatured by heating for 3 minutes at 96° C., and the mixture was then incubated at 65° C. for 10 minutes to allow hybridization of target and oligonucleotide probe. Binding of the biotinylated oligonucleotide to streptavidin-coated beads (Bangs Laboratories) was carried out by supplementing the reaction mixture with 5×10⁴ beads in a final volume of 15 μL and incubated the mixture for 10 minutes at room temperature.

### Binding of Fluorophore

To a 500 μL aliquot of a working stock solution of fluorophore was added 10 μL of hybridized target-probe bead suspension and the mixture was incubated at room temperature for a minimum of 5 minutes. The working stock solution of fluorophore was prepared from the original preparation supplied by the manufacturer as follows: picogreen was diluted 1:10,000 with TE buffer, SYBR GREEN was diluted 1:200 with TE buffer, thiazole orange (Aldrich Chemical Company, Milwaukee, Wis.) 1 μg/mL in TE buffer, TOPRO-1, TOTO-1, YOPRO-1 and YOYO-1, all 0.5 μM in TE buffer.

### Nuclease Protection

After hybridization of bead-immobilized oligonucleotide probes (SEQ ID NO:1) to target DNA (SEQ ID NO:2), an 8 μL aliquot of the suspension was combined with 8 μL of 2×51 nuclease buffer or 2× mung bean nuclease buffer (Promega, Madison, Wis.), digested with 1 unit of S1 nuclease or mung bean nuclease, and incubated at 30° C. for 30 minutes. To the reaction mixture was added, a 1:10 dilution of streptavidin-conjugated phycoerythrin fluorophore in TE buffer to a final volume of 24 μL.

### Flow Cytometry

Flow cytometric analysis was performed using an Ortho CYTORONABSOLUTE® Flow Cytometer with Immunocount 2.2 software. Parameters for particle analysis were determined for each lot of bead-immobilized oligonucleotide. Gains and amplifiers for forward-angle scattering and right-angle scattering were setup such that beads of each size could be detected and resolved by the instrument. Fluorescence gain and amplifier settings in the CYTORONABSOLUTE® Flow Cytometer were adjusted to optimize hybridization-mediated fluorescence detection. Cluster analysis was used to determine the mean-peak channel fluorescence, since it allows thresholds of both fluorescence and another parameter (e.g., forward scattering or right scattering) to be preset, therefore permitting uniform criteria to be applied to different samples being analyzed. This procedure eliminates background noise resulting from particles presenting highly scattered fluorescence channel values.

The examples presented below utilize DNA as target nucleic acid. This is for illustrative purposes only. Wherever necessary, the methods of the present invention can be adapted readily for RNA targets, as would be known to the skilled practitioner.

### Example 1

### Detection of Target DNA Hybridized to Particle-Immobilized Oligonucleotide: DNA Binding Fluorophores

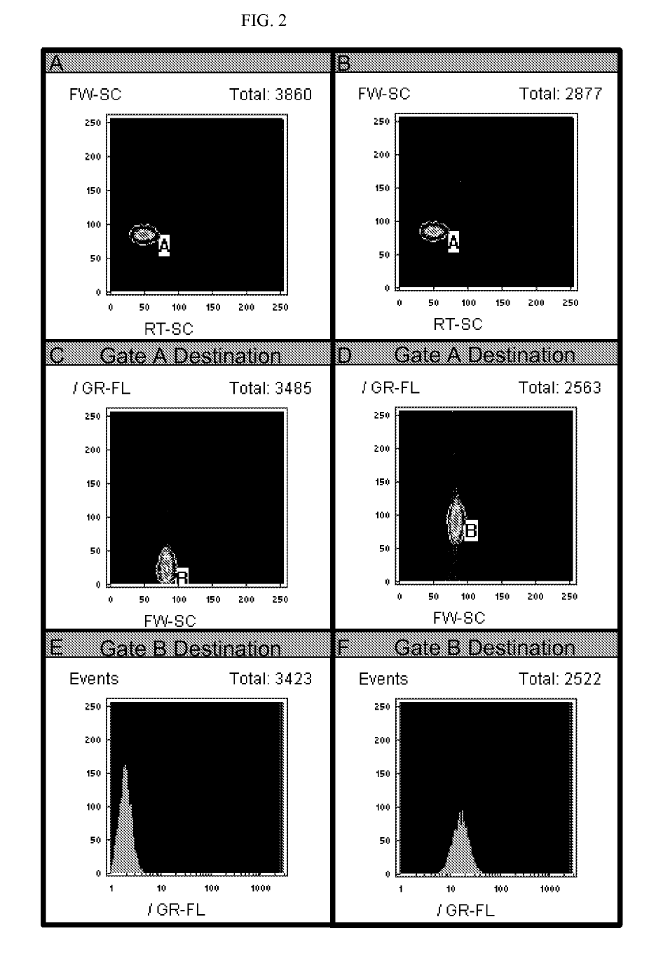

Fluorophores that bind to dsDNA were used to detect hybridized target DNA. The target DNA in this example (SEQ ID NO:2) was hybridized to bead-immobilized probe (SEQ ID NO:1) in the presence of excess calf thymus DNA. Fluorophore was combined with hybridized target-bead suspension and analyzed by flow cytometry. Results are illustrated in FIGS. 2A-F using thiazole orange as the dsDNA binding fluorophore. The forward-angle scattering (FW-SC) x right-angle scattering (RT-SC) pattern of beads incubated with 1 μg calf thymus DNA in the absence (FIG. 2A) and presence (FIG. 2B) of target DNA is shown. Light-scatter gating of the beads by means of an image analysis software algorithm allows the analysis of select particles within a narrow range of FW-SC x RT-SC values, thus, eliminating particles outside the size range. The gated group of particles has the FW-SC x Green Fluorescence (GR-FL) pattern illustrated in FIGS. 2, C and D, with thiazole orange as the dsDNA binding fluorophore, where further gating selects for the events used to calculate a mean channel fluorescence value. The group of particles selected in FIGS. 2, C and D is represented in a fluorescence histogram in FIGS. 2, E and F, where it can be seen that the distribution averages of a negative control (no target DNA) and positive (1000 fmoles of target) are clearly separated. Table 1 below shows the mean-peak channel fluorescence (MCF) for seven different fluorophores in the presence of CTDNA with and without added target DNA. Hybridizations were performed in a mixture containing 4×10⁴ beads comprising oligonucleotide probe (SEQ ID NO:1) immobilized thereto, with or without target (SEQ ID NO:2, 1000 fmoles) in presence of 1 microgram of CTDNA and 0.15M potassium chloride, 0.01M Tris, and 1 mM (EDTA), pH 8.3, in a final volume of 10 microliters. The DNA was denatured at 96° C. for 3 minutes and hybridized at 65° C.

TABLE 1

| Fluorophore | no target | 1000 fmoles target |
|---|---|---|
| Thiazole Orange | 22.5 | 87.4 |
| Picogreen | 27.1 | 103.7 |
| Sybrgreen | 15.8 | 51.5 |
| TO-PRO-1 | 19.3 | 101.6 |
| TOTO-1 | 13 | 49.1 |
| YO-PRO-1 | 20.6 | 59.8 |
| YOYO-1 | 36 | 63.6 |
| Phycoerythrin | 21.5 | 34.7 |

There is no need to separate the particles associated with hybridized target and oligonucleotide from DNA free in solution as light-scatter gating of the particles by image analysis software allows analysis of a select group of those particles that present a cohesive, narrow range of forward angle scattering x right angle scattering values; limiting the analysis to only those particles of interest.

### Example 2

### Nuclease Protection

Hybridization of biotinylated particle-immobilized oligonucleotide probes (Oligo-1A) to target oligonucleotide (SEQ ID:2) protected the probe from hydrolysis by single strand specific DNA endonuclease. As in Example 1, particle-immobilized oligonucleotide and CTDNA were incubated together in the presence and absence of 1000 femtomoles of target DNA, followed by incubation with S1 nuclease. Biotin was released upon endonuclease hydrolysis of the oligonucleotide probe unless it was protected from hydrolysis by hybridization with target DNA.

An 8 microliter aliquot of streptavidin-linked fluorophore (streptavidin-phycoerythrin from Molecular Probes), diluted 1:10 in TE buffer, was added to 16 microliters of nuclease-treated sample, and incubated for 10 minutes at room temperature. Binding of streptavidin-linked fluorophore served as reporter for intact bead-linked oligonucleotide. The mixture was analyzed using flow cytometry. The mean channel fluorescence of phycoerythrin without target was 21.5, with 1000 femtomoles of target it was 34.7.

### Example 3

### Quantification of Target DNA: DNA Binding Fluorophore

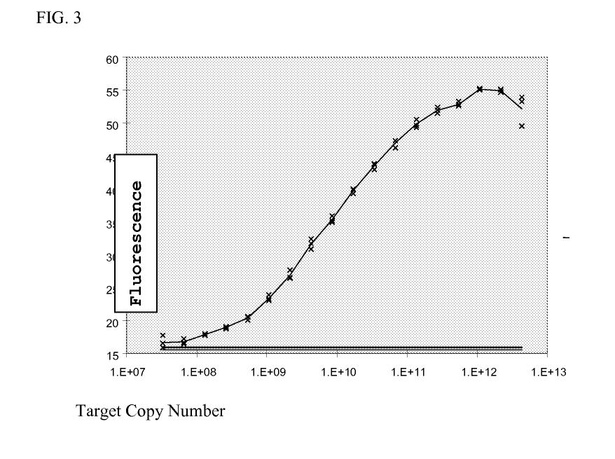

The fluorescence of the fluorophore, sybr green, bound to the hybrid of target DNA (SEQ ID NO:2) and bead-immobilized oligonucleotide (SEQ ID NO:1) is shown as a function of the copy number of the target in FIG. 3.

The fluorescence signal associated with bead-immobilized oligonucleotide is monotonically dependent on the concentration, demonstrating the ability to quantitatively determine the amount of target DNA over a concentration range of about 6 orders of magnitude. As little as 25.7 picograms of target dsDNA corresponding to 440 attomoles, or about 2.5×10⁸ copies of the 90-mer target, was clearly detected in a background of 0.8 μg non-specific calf thymus DNA. Thus the target DNA was readily detectable in the presence of about a 3.11×10⁴-fold excess of non-specific DNA. The results also show that the method is very sensitive over the concentration range; a two-fold increase in target DNA concentration was readily detectable within a concentration range between about 1.25×10⁸ to 1.09×10¹² copies of the target in a volume of 8 microliters. The average fluorescence obtained with thiazole orange, TOPRO-1, TOTO-1, YOPRO-1, YOYO-1, and picogreen, in each case, was also proportional to the concentration of the target DNA (data not shown).

### Example 4

### Quantification of Target DNA: dsDNA Binding Fluorophore

In an alternative embodiment, a soluble, biotinylated oligonucleotide probe was allowed to hybridize to its target DNA in solution. The target DNA-biotin-oligonucleotide probe hybrid was then allowed to bind to streptavidin-beads. To the suspension was then added 500 microliters of a 1:200 dilution of picogreen and incubated 2 min at room temperature. The suspension was introduced into the flow cytometer. The calculated particle-associated mean channel fluorescence was proportional to the concentration of target DNA in the sample (data not shown).

### Example 5

### Quantification of Target DNA: Nuclease Protection

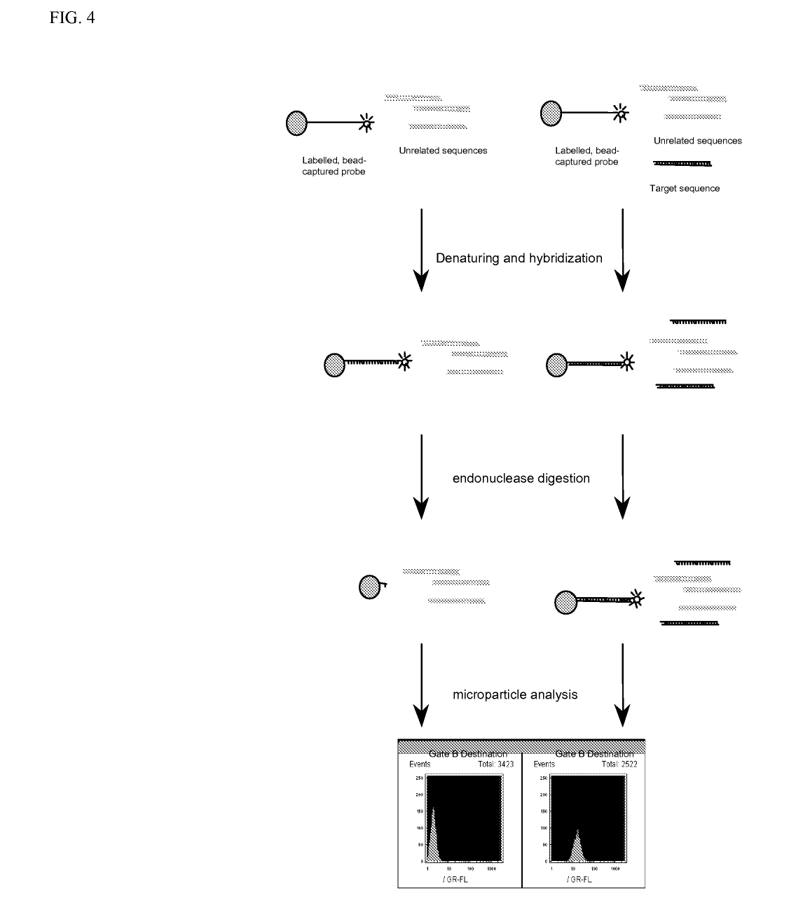

FIG. 4 shows a schematic of a nuclease protection-based assay for detection of particle-associated target nucleic acid.

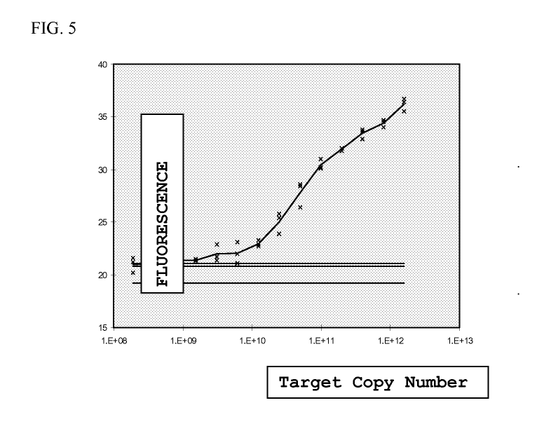

Protection of oligonucleotide probe from nuclease S1 digestion was proportional to the amount of target DNA. FIG. 5 shows the average fluorescence signal as a function of increasing single-stranded target DNA concentration. As little as 40.96 femtomoles (about 2.5×10¹⁰ copies) of the 90 base-pair DNA target (SEQ ID NO:2) was detected in about a 100-fold excess of non-target CTDNA.

### Example 6

### Detection of PCR Amplification Products: DNA Binding Fluorophore

To quantitatively measure low-abundance nucleic acids, PCR amplification of target DNA is frequently monitored as a function of amplification cycle number; the number of amplification cycles required to detect product being proportional to the target copy number.

In this example, 10 copies of target DNA were amplified from a plasmid-cloned DNA insert (SEQ ID NO. 3) using PCR. Target DNA (10 copies, SEQ ID:NO:3) was amplified in a volume of 100 microliters of an admixture containing CT (calf thymus) DNA, 5 micromolar NaOH, 4 milimolar MgCl₂, 18 mM Tris buffer, 54 mM KCl, 0.4 mM each primer (SEQ ID NO: 4 and SEQ ID NO: 5), 0.3 mM each dNTP, 0.108 microgram/microliter gelatin, 0.725 mM EDTA, 40 micromolar DTT, 9.5% glycerol, 0.02% Tween 20, 0.02% Nonidet P40.

### Target DNA

The target DNA having the following sequence consisted of a DNA fragment cloned in pUC18:

SEQ ID:NO: 3:
5′-CTGCAGGCGC CAGCGTGGAC CATCAAGTAG TAATGAACGC
ACGGACGAGG ACATCATAGA GATTACACCT TTATCCACAG
TTCTCGGTCT AACGCAGCAG TCAGTGTATC AGCACCAGCA
TCCGTAGTGA GTCTTCAGTG TCTGCTCCAG GATCGTGGCG
CTGCAG-3′

The underlined sequences correspond to the region amplified from the target DNA using the PCR primers described bellow (SEQ ID:NO:4 AND SEQ ID:NO:5).

### PCR Primers

Primers used for target DNA amplification were synthetically prepared oligonucleotides according to the sequences:

SEQ ID NO: 4: (forward primer)
5′-CGCCAGCGTG GACCATCAAG TAGTAA-3′
SEQ ID NO: 5: (reverse primer)
5-′CACGATCCTG GAGCAGACAC TGAAGA-3′

PCR was carried out using standard thermal cycling protocols (cycles 1-5: 30s at 96° C.: 60s at 68° C.; cycles 6-40: 15s at 96° C.; 60s at 68° C.), terminating the reactions after either 5, 10, 15, 20, 25, 30, 35, and 40 cycles in the Perkin Elmer 9600 PCR System Thermocycler.

### Hybridization

After amplification, 10 μL of the PCR admixture were combined with 10 microliters of a suspension containing about 10⁵ 3.5 micron diameter particles (Bangs Labs, Fishers, Ind.), previously coupled to the oligonucleotide probe (SEQ ID: NO: 6) suspended in 2× hybridization buffer (0.3M potassium chloride, 0.02M Tris and 2 mM EDTA, pH 8.3), and the mixture was then heated for 3 minutes at 96° C., then allowed to cool to 65° C. and incubated for ten minutes.

### Microparticle-Immobilized DNA Probe

The oligonucleotide probe immobilized on the particle has the sequence:

SEQ ID NO: 6:
5′-CTGCGTTAGA CCGAGAACTG TGGATAAAGG-3′

SEQ ID NO:6: was modified by covalent attachment of biotin to the 3′ terminus. The probe was allowed to bind to streptavidin-coated particles, 3.5 micrometer in diameter (Bangs Laboratories) using standard protocols.

### DNA Staining

DNA staining was carried out by combining 500 μL of a 1:200 dilution of concentrated picogreen fluorescent dye with 20 μL of bead suspension comprising hybridized target and incubating the suspension for a minimum of 2 minutes at room temperature. Samples were next analyzed with the CYTORONABSOLUTE® flow cytometer.

### Results

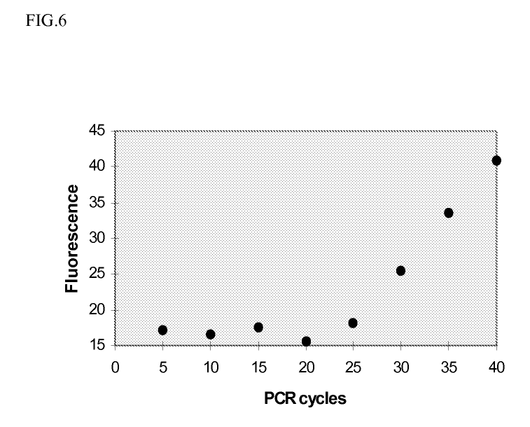

PCR product was measured as mean channel fluorescence derived from picogreen bound to hybridized target and particle-immobilized oligonucleotide dsDNA. In FIG. 6, it can be seen that the mean channel fluorescence increased with increasing PCR cycle number after 30 cycles demonstrating quantitative detection of PCR amplification product.

### Example 7

### Laser Scanning Cytometry

Laser scanning cytometry can be used in practicing the present invention. In a laser scanning cytometer, such as the Compucyte Laser Scanning Cytometer, an optical detector moves past substances dispersed, usually uniformly, on a surface in two spatial dimensions, for instance, on the surface of a microscope slide. This differs from flow microfluorimetry wherein particles move past a detector in one dimension (substantially one at a time in a moving stream).

Particles comprising a target-specific oligonucleotide probe must be large enough to be resolved by the detection system used in the laser scanning cytometer. Particles are preferably greater than or equal to about 0.3 microns, more preferably between about 1 to about 5 microns. The particles, sample comprising target nucleic acid and a compound that produces a detectable fluorescence when bound to dsDNA are combined to form a mixture. The mixture is diluted sufficiently to allow uniform dispersion of the particles on a microscope slide. The laser scanner moves over the slide and uses light scattering and/or fluorescence of the particles to trigger measurement of detectable fluorescence from a compound that is bound to hybridized target and particle-immobilized oligonucleotide. The laser scanning cytometer differentiates a plurality of distinct target-specific particles by the specific light scattering and/or fluorescence characteristics of the target-specific particles.

### Example 8

### DNA Arrays

In laser scanning cytometry and flow microfluorimetry, analyte detection relies on spatial resolution. In flow cytometry, resolution is one dimension. In laser scanning cytometry resolution is in two dimensions.

A DNA chip comprising oligonucleotide arrays represents a two-dimensional separation device. Methods for preparing such arrays are described in WO9818961 and in the U.S. Patents noted in the Background section.

In operation, a range of measurements, x1, is made in one direction, the x direction, and a range of measurements, y1, is made in a direction orthogonal to x, the y direction. Events detected as x1y1 are known to be associated with a particular target-specific probe. Similarly, x2y2 measurements are known to be associated with a probe for the same target or for a different target, and so on.

DNA chips or slides comprising oligonucleotide arrays can be prepared by depositing and anchoring oligonucleotides directly to the surface or indirectly through chemical linkers or other immobilizing agents, all using techniques known in the art and found in WO 9818961, as well as the US Patents noted heretofore. They are immobilized in discrete xy locations on one surface of a chip or slide. Each xy locus, or spot, is specific for any desired target nucleic acid. Multiple spots specific to a single target can be located on the chip or slide to effect assay replication (replicate spots). Any desired number of target-specific spots and replicate spots can be used. Preferably, the chip or slide comprises between about 5 to about 20 target-specific spots and about the same number of replicate spots, more preferably the chip or slide comprises between about 100 to about 1000 target-specific spots and 10 to 100 replicate spots for each target. The DNA chip or slide is contacted with sample, and a compound that produces a detectable fluorescence signal when bound to dsDNA. The target-correlated fluorescence originating from the spots, that is, each specific xy location on the chip or slide is measured, for example, using a laser scanning device. The fluorescence signal is related to the presence or amount of the specific target nucleic acid.

In particular, a DNA chip having about 100 target-specific loci and about 10 replicate loci to SEQ. ID NO. 1, which oligonucleotide sequences are printed on silylated slides (CEL Associates). The print spots are about 125 μm in diameter and are spaced 300 μm apart from center to center. About 10 replicate probes to irrelevant sequence (for example, probes to plant genes where mammalian sequences are detected) are also printed on the slides. Printed glass slides are treated with sodium borohydrate solution (0.066M NaBH4, 0.06M Na AC) to ensure amino-linkage of probes to the slides. The slides are then boiled in water for 2 minutes to denature the cDNA. A biological containing from about 30 to about 30,000 target nucleic molecules is heated to 99° C. for 5 minutes, then pre-cooled before hybridization, in this case, held at room temperature for 5 minutes, and then applied to the slides. The slides are covered with glass cover slips, sealed with DPX (Fluka) and hybridized at 60° C. for 4-6 hours. At the end of hybridization slides are cooled to room temperature. The slides are washed in 1×SSC, 0.2% SDS at 55° C. for 5 minutes, 0.1×SSC, 0.2% SDS at 55° C. for 5 minutes. The slides are stained by contacting the entire surface with a 1:200 dilution of concentrated picogreen fluorescent dye and incubating for a minimum of 2 minutes at room temperature. After a quick rinse in 0.1×SSC, 0.2% SDS, the slides are air-blown dried and ready for scanning. Arrays are scanned for picogreen dye fluorescence using the ScanArray 3000 (General Scanning, Inc.). ImaGene Software (Biodiscovery, Inc.) is subsequently used for quantitation. The intensity of each spot is corrected by subtracting the immediate surrounding background.

The fluorescent signal is related to the presence or amount of the specific target nucleic acid and the slide has performed 10 replicates of this assay with mean of fluorescence from replicate test spots compared using statistical methods to mean of fluorescence from replicate probes to irrelevant sequence.

The above methods and procedures are repeated using a DNA chip having about 100 target-specific loci and about 10 replicate loci to SEQ. ID NO. 1, SEQ. ID NO. 2, SEQ. ID NO. 3, SEQ. ID NO 4, SEQ. ID NO. 5 and SEQ. ID NO.6.

### Example 9

### Detection of PCR Amplification Products: Homogeneous Method

The purpose of this Example is to perform PCR thermocycling with IPC—IP beads in the PCR mix. Addition of DNA stain follows thermocycling.

The materials and procedures of Example 6 are employed, using the conditions, primers and probes identified below. Target DNA (10 copies), SEQ. ID No. 3, is amplified in a volume of 100 microliters using suitable PCR protocols that includes components of a PCR admixture, 0.25 microliter of 10⁵ oligonucleotide-bound 1.7 micron beads. Beads used are poly[styrene-co(p-vinylbenzylthio)proprionic acid] 95:5 molar ratio beads prepared as described in U.S. Pat. Nos. 5,149,737; 5,210,289 and 5,278,267, and the appropriate concentration of a DNA-binding fluorophore, in this case, 500 microliter of a 1:200 dilution of concentrated picogreen, which is added following the thermocycling. Amplification reactions are set up in PCR conditions of Example 6, except 10⁵ oligonucleotide-bound microparticles is included in the admixture and a temperature cycle allowing for hybridization after the PCR cycles is added. The presence of the amplified target DNA in the resulting mixture is then determined by flow cytometry using, for example, the CYTORONABSOLUTE® flow cytometer.

### Target DNA

A target DNA having the following sequence is cloned in pUC18:

SEQ ID NO. 3:
5′-CTGCAGGCGC CAGCGTGGAC CATCAAGTAG TAATGAACGC
ACGGACGAGG ACATCATAGA GATTACACCT TTATCCACAG
TTCTCGGTCT AACGCAGCAG TCAGTGTATC AGCACCAGCA
TCCGTAGTGA GTCTTCAGTG TCTGCTCCAG GATCGTGGCG
CTGCAG-3′

### Amplification and Hybridization Reactions

Using PCR conditions and primers identified below, the sequences underlined in SEQ ID NO.3: are amplified using modified amplification protocols (cycles 1-5: 30s at 96° C.: 60s at 68° C.; cycles 6-40: 15s at 96° C.; 60s at 68° C.; cycle 41: 5 minutes at 72° C.; cycle 42: 3 minutes at 96° C.; cycle 43: 60s at 50° C.) in the PE9600 Thermocycler.

SEQ ID NO. 4: (forward primer)
5′-CGCCAGCGT GGACCATCA AGTAGTAA-3′
SEQ ID NO. 5: (reverse primer)
5-′CACGATCCT GGAGCAGAC ACTGAAGA-3′

The oligonucleotide probe immobilized on the particle has the sequence:

SEQ ID NO. 6:
5′-CTGCGTTAG ACCGAGAAC TGTGGATAA AGG-3′

SEQ ID NO.6: is modified by covalent attachment to the surface of polystyrene particles, 1 micrometer in diameter using standard protocols.

### DNA Staining

DNA staining is carried out by combining 500 μL, of a 1:200 dilution of concentrated picogreen with 20 μL of the above-identified PCR bead suspension comprising hybridized amplified target, and incubating the suspension for a minimum of 2 minutes at room temperature

### Results

PCR product is measured as mean channel fluorescence derived from picogreen bound to hybridized target and particle-immobilized oligonucleotide dsDNA.

### Example 10

### Detection of PCR Amplification Products: Homogeneous Method

The purpose of this Example is to perform PCR thermocycling with beads and DNA dye in the PCR mix. The materials and procedures of Example 9 are employed except that 2.5 microliters of concentrated thermostable dye (for example, SYBR Green I dye, Molecular Probes, Eugene, Oreg.) is added to the PCR suspension containing target, prior to thermocycling. Other thermostable dyes that can be used include ethidium bromide and propidium iodide.

### Results

PCR product is measured as mean channel fluorescence derived from SYBR Green bound to hybridized target and particle-immobilized oligonucleotide dsDNA.

### Example 11

### Detection of PCR Amplification Products: Homogeneous Method

The purpose of this Example is to perform PCR thermocycling in the presence of thermostable DNA dye in the PCR mix. Addition of microbeads follows thermocycling. The materials and procedures of Example 9 are employed with 2.5 microliters of a concentrated thermostable SYBR Green dye added to the PCR target suspension prior to thermocycling. Other thermostable dyes that can be used include ethidium bromide and propidium iodide.

### DNA Hybridization

Following thermocycling, 10 microliters of 10⁵ oligonucleotide-bound microparticles (1 to 10 microns in size) suspended in 2× hybridization buffer (0.3M potassium chloride, 0.02M Tris and 2 mM EDTA, pH 8.3), are added to 10 microliters of the amplified product-dye admixture, and heated for 3 minutes at 96° C., then allowed to cool to 65° C. to allow hybridization.

### Analysis

The hybridized samples are supplemented with 500 μL of 1 mM Tris, 10 mM EDTA buffer and analyzed with for example, the CYTORONABSOLUTE® flow cytometer.

### Results

PCR product is measured as mean channel fluorescence derived from SYBR Green bound to hybridized target and particle-immobilized oligonucleotide dsDNA.

### Example 12

### Detection of Immobilized Target DNA Hybridized to Oligonucleotide with Detection Via DNA Binding Fluorophores

In this example, an article is identified or authenticated by immobilizing target DNA to the article, simultaneously contacting the article with both a complementary oligonucleotide and a compound which is capable of binding to duplex nucleic acid and which upon binding or being bound thereto is capable of producing a detectable signal. In this example, a marking pen is used to deliver both complementary oligonucleotide and an intercalating dye.

A target DNA (Target 1) comprises SEQ ID NO. 1, as follows:

5′-TTTCCAAGTA AGCAATAACG TCAGCTCTTT CTTGTGGCTT
CTTCATACCA GCGAAAGACA TCTTAGTACC TGGCATGAAC
TTCTTTGGGT-3′.

Target 1 is associated with the surface of an article using the method of Okamato, et al. (“Microarray fabrication with covalent attachment of DNA using Bubble Jet technology”, Nature Biotechnology 18, 438-441 (2000)). Articles are printed in a 10×10 array of possible locations such that the locations used are in the shape of the letters “A”, “T”, “C”, or “G”.

A detector oligonucleotide (Detector 1) comprises SEQ ID NO:2, as follows:

5′-ACCCAAAGAA GTTCATGCCA GGTACTAAGA
TGTCTTTCGC TGGTATGAAG AAGCCACAAG AAAGAGCTGA
CGTTATTGCT TTGGAAA-3′.

Target 1 is detected using a marking pen that delivers Detector 1 and approximately 0.5 μM intercalating dye selected from the list of Thiazole Orange, Picogreen, Sybrgreen, TO-PRO-1, YO-PRO1, or YOYO-1. For each article to be authenticated, the pen delivers approximately 1 pmole of Detector 1 in the presence of 1 microgram of CTDNA and 0.15M potassium chloride, 0.01M Tris, and 1 mM (EDTA), pH 8.3, in an approximate volume of 10 μL over the array area on the article.

Hybridization of Detector 1 to Target 1 is detected by illumination of the array area with an excitation wavelength of light matched to the dye selected, and visualized through an appropriate optical filter matched to the fluorescence of said dye, or preferably using a handheld reader designed to deliver said light and measure said fluorescence location-by-location within the array.

The skilled artisan recognizes that the methods of this example may use other DNA sequences for target and detector DNA, including DNA from natural sources, that other methods are suitable for both associating the target DNA with the article, and that the other methods are available for binding and detection of the intercalating dye to the target/detector heteroduplex. Additionally, the requirement for detector oligonucleotide to match target nucleic acid provides for “lock & key” assurance. Also, the present example illustrates that an additional level of assurance is created by the spatial information revealed.

### Example 13

### Detection of Target DNA Hybridized to Detector Oligonucleotide Immobilized to Signature Array Elements

This example shows a method for authenticating an article by determining the presence of a code comprising at least two target nucleic acid sequences upon contacting the article with both a detector oligonucleotide and an intercalating dye. Further, in this example, the target DNA is associated with the article at discrete locations and in morphologically distinct shapes.

A target DNA (Target 2) comprises the same number of nucleotides as Target 1, but no sequence identity at any position. A detector oligonucleotide (Detector 2) is synthesized to comprise sequence complimentary to Target 2.

Target 1 and Target 2 are printed on articles for authentication by printing one or the other target DNA in each of two locations on the article (i.e., four possible combinations, as follows: Position 1=Target 1, Position 2=Target 1; Position 1=Target 1, Position 2=Target 2; Position 1=Target 2, Position 2=Target 1; and, Position 1=Target 2, Position 2=Target 2). For each group of articles to share the same code, one of the characters A-Z or numbers 0-9 is selected for each of two locations (36 combinations), and each character at each location is printed in one of 3 discernable sizes. The skilled artisan recognizes that this simple system yields 7776 possible codes from just two target DNA's.

Target 1 and/or Target 2 is detected using a marking pen that delivers complementary Detector DNA and approximately 0.5 μM intercalating dye as described in Example 1.

### Example 14

### Detection of Target DNA in Combination with a Signature Array Using Detector DNA and a DNA Binding Fluorophore

Streptavidin Coated Polystyrene Particles, 0.5% w/v, are purchased from Spherotech, Inc., 1840 Industrial Dr., Suite 270, Libertyville, Ill. 60048. Target DNA of the above Example 2 is modified by linking biotin to the 5′ terminus through two tetraethylene glycol (TEG-TEG) spacers with and without an aminodiol (ADL) linker at the 3′ terminus for attachment to microparticles. To prepare the microparticles of each cluster, biotinylated target DNA is incubated at ambient temperature for 10 minutes with 5×10⁴ beads per 10 μL in 0.15M potassium chloride, 0.01M tris(hydroxymethyl)aminomethane (Tris), 1 mM ethylenediaminetetraacetic acid (EDTA), pH 8.3, according to Table 2.

TABLE 2

| Cluster | Particle | |
|---|---|---|
| # | Size | DNA |
| 1 | 0.3-0.39 μm | Target 1 |
| 2 | 0.4-0.6 μm | Target 1 |
| 3 | 0.7-0.9 μm | Target 1 |
| 4 | 1.5-1.9 μm | Target 1 |
| 5 | 2.0-2.9 μm | Target 1 |
| 6 | 3.0-3.9 μm | Target 1 |
| 7 | 4.0-4.9 μm | Target 1 |
| 8 | 5.0-5.9 μm | Target 1 |
| 9 | 6.0-8.0 μm | Target 1 |
| 10 | 8.0-13.9 μm | Target 1 |
| 11 | 14.0-17.9 μm | Target 1 |
| 12 | 18.0-23.0 μm | Target 1 |
| 13 | 0.3-0.39 μm | Target 2 |
| 14 | 0.4-0.6 μm | Target 2 |
| 15 | 0.7-0.9 μm | Target 2 |
| 16 | 1.5-1.9 μm | Target 2 |
| 17 | 2.0-2.9 μm | Target 2 |
| 18 | 3.0-3.9 μm | Target 2 |
| 19 | 4.0-4.9 μm | Target 2 |
| 20 | 5.0-5.9 μm | Target 2 |
| 21 | 6.0-8.0 μm | Target 2 |
| 22 | 8.0-13.9 μm | Target 2 |
| 23 | 14.0-17.9 μm | Target 2 |
| 24 | 18.0-23.0 μm | Target 2 |

In order to assure classification of the particles to the correct cluster, signature arrays are constructed of clusters that are comprised of alternating use of size range and Target DNA, according to Table 3 as follows:

TABLE 3

| Cluster # | Particle Size | | Set 1 | Set 2 |
|---|---|---|---|---|
| 1 | 0.3-0.39 | μm | None or 1X to | None |
| | | | 4X | |
| 2 | 0.4-0.6 | μm | None | None or 1X to |
| | | | | 4X |
| 3 | 0.7-0.9 | μm | None or 1X to | None |
| | | | 4X | |
| 4 | 1.5-1.9 | μm | None | None or 1X to |
| | | | | 4X |
| 5 | 2.0-2.9 | μm | None or 1X to | None |
| | | | 4X | |
| 6 | 3.0-3.9 | μm | None | None or lx to |
| | | | | 4X |
| 7 | 4.0-4.9 | μm | None or lx to | None |
| | | | 4X | |
| 8 | 5.0-5.9 | μm | None | None or lx to |
| | | | | 4X |
| 9 | 6.0-8.0 | μm | None or lx to | None |
| | | | 4X | |
| 10 | 8.0-13.9 | μm | None | None or 1X to |
| | | | | 4X |
| 11 | 14.0-17.9 | μm | None or 1X to | None |
| | | | 4X | |
| 12 | 18.0-23.0 | μm | None | None or 1X to |
| | | | | 4X |
| 13 | 0.3-0.39 | μm | None or 1X to | None |
| | | | 4X | |
| 14 | 0.4-0.6 | μm | None | None or 1X to |
| | | | | 4X |
| 15 | 0.7-0.9 | μm | None or 1X to | None |
| | | | 4X | |
| 16 | 1.5-1.9 | μm | None | None or 1X to |
| | | | | 4X |
| 17 | 2.0-2.9 | μm | None or 1X to | None |
| | | | 4X | |
| 18 | 3.0-3.9 | μm | None | None or 1X to |
| | | | | 4X |
| 19 | 4.0-4.9 | μm | None or 1X to | None |
| | | | 4X | |
| 20 | 5.0-5.9 | μm | None | None or 1X to |
| | | | | 4X |
| 21 | 6.0-8.0 | μm | None or 1X to | None |
| | | | 4X | |
| 22 | 8.0-13.9 | μm | None | None or 1X to |
| | | | | 4X |
| 23 | 14.0-17.9 | μm | None or 1X to | None |
| | | | 4X | |
| 24 | 18.0-23.0 | μm | None | None or 1X to |
| | | | | 4X |

Thus, 10¹² different signatures can be constructed from just these 12 different bead sets and 2 different target DNAs(!). Microparticles comprising clusters of a selected signature are associated with a liquid product. To detect the presence of said signature, the microparticles are concentrated by centrifugation, then mixed with detector DNA at a final suspension containing about 10⁵ particles per 10 μL in 0.3M potassium chloride, 0.02M Tris and 2 mM EDTA, pH 8.3. The mixture is then heated for 3 minutes at 96° C., then allowed to cool to 65° C. and incubated for ten minutes.

DNA staining is carried out by combining 500 μL of a 1:200 dilution of concentrated picogreen fluorescent dye with 20 μL of bead suspension comprising hybridized target and incubating the suspension for a minimum of 2 minutes at room temperature. Samples are analyzed with a flow cytometer. Thus, any measured fluorescence signal is generated from particle-immobilized target/detector DNA heteroduplex, and a signature array is revealed.

### Example 15

### Laser Scanning Cytometry

Laser scanning cytometry can be used in practicing the present invention. In a laser scanning cytometer, such as the Compucyte Laser Scanning Cytometer, an optical detector moves past substances dispersed, usually uniformly, on a surface in two spatial dimensions, for instance, on the surface of a microscope slide. This differs from flow microfluorimetry wherein particles move past a detector in one dimension (substantially one at a time in a moving stream).

Particles comprising a target-specific oligonucleotide probe must be large enough to be resolved by the detection system used in the laser scanning cytometer. Particles are preferably greater than or equal to about 0.3 microns, more preferably between about 1 to about 5 microns. The particles, sample comprising target nucleic acid and a compound that produces a detectable fluorescence when bound to dsDNA are combined to form a mixture. The mixture is diluted sufficiently to allow uniform dispersion of the particles on a microscope slide. The laser scanner moves over the slide and uses light scattering and/or fluorescence of the particles to trigger measurement of detectable fluorescence from a compound that is bound to hybridized target and particle-immobilized oligonucleotide. The laser scanning cytometer differentiates a plurality of distinct target-specific particles by the specific light scattering and/or fluorescence characteristics of the target-specific particles.

The present invention has been described in detail in respect to particular preferred embodiments. It will be understood that variations and modifications can be effected without departing from the scope and spirit of the invention. The entire contents of all cited patents, patent applications, and non-patent disclosures are expressly incorporated herein by reference.

We claim:

1. A method for authenticating a product by determining the presence of a target nucleic acid comprising the steps of:
a) associating an authentication code comprising the target nucleic acid with the article to be authenticated;
b) contacting the article with an oligonucleotide probe comprising a nucleic acid sequence complementary to at least a portion of the target nucleic acid to form a duplex heterodimer; and
c) determining the presence of the authentication code by contacting the duplex heterodimer formed by said target nucleic acid and oligonucleotide probe to a reporter compound which is capable of binding to duplex nucleic acid and which upon binding or being bound thereto is capable of producing a detectable signal.

2. The method of claim 1, wherein the target nucleic acid is incorporated into the formulation of the product.

3. The method of claim 1, wherein the target nucleic acid is immobilized on the surface of the product.

4. The method of claim 3, wherein immobilization is performed in a specified morphology or symbology wherein revealing the presence of the target oligonucleotide also reveals a specific shape or symbol and/or size at each location.

5. The method of claim 1, wherein the target nucleic acid is on the product packaging.

6. The method of claim 1, wherein the target nucleic acid is contained in an authentication pouch associated with the product.

7. The method of claim 1, wherein steps b) and c) are performed sequentially.

8. The method of claim 7, wherein the oligonucleotide probe and the reporter compound are contained in a detecting fluid for spotting in-situ on immobilized target nucleic acid.

9. The method of claim 1, wherein the reporter compound is an intercalating dye.

10. A method for authenticating a product by determining the presence of an authentication code comprising target nucleic acid, said method comprising the steps of:
a) immobilizing the target nucleic acid to a substrate, wherein the substrate is a microparticle;
b) associating said substrate with the product to be authenticated;
c) contacting the substrate to an oligonucleotide probe comprising a nucleic acid sequence complementary to at least a portion of the target nucleic acid to form a duplex heterodimer; and
d) determining the presence of the authentication code by contacting the duplex heterodimer formed by said target nucleic acid and oligonucleotide probe to a reporter compound which is capable of binding to duplex nucleic acid and which upon binding or being bound thereto is capable of producing a detectable signal.

11. The method of claim 10, wherein the microparticle comprises a distinct first compound or plurality of compounds capable of producing a distinct fluorescence signal corresponding to the microparticle.

12. The method of claim 10, wherein the microparticle is capable of scattering electromagnetic radiation of wavelength greater than or equal to about 200 nm.

13. The method of claim 12, wherein the microparticle comprises a distinct first compound or plurality of compounds capable of producing a distinct fluorescence signal corresponding to the microparticle.

14. The method of claim 10, wherein the target nucleic acid and the substrate are incorporated into the formulation of the product.

15. The method of claim 10, wherein the target nucleic acid and the substrate are immobilized on the surface of the product.

16. The method of claim 10, wherein the target nucleic acid and the substrate are on the product packaging.

17. The method of claim 10, wherein the target nucleic acid and the substrate are contained in an authentication pouch associated with the product.

18. The method of claim 10, wherein steps c) and d) are performed sequentially.

19. The method of claim 10, wherein the oligonucleotide probe and the reporter compound are contained in a detecting fluid for spotting in-situ on immobilized target nucleic acids.

20. The method of claim 10, wherein the reporter compound is an intercalating dye.

21. The method of claim 10, wherein immobilization is performed in a specified morphology or symbology wherein revealing the presence of the target oligonucleotide also reveals a specific shape or symbol and/or size at each location.

22. The method of claim 10, wherein the oligonucleotide probe of step c is covalently labeled with a reporter that generates a detectable signal, making step d) unnecessary.

23. The method of claim 22, wherein the reporter is a fluorescent moiety.

24. The method of claim 23, wherein the reporter is a molecular beacon.

25. The method of claim 10, wherein the authentication code further comprises the count or relative count of substrates per cluster, said cluster being a grouping of substrates sharing one or more discretely measurable properties.

26. A method for authenticating a product by determining the presence of an authentication code comprising target nucleic acid, said method comprising the steps of:
a) immobilizing the target nucleic acid to the product;
b) contacting said target nucleic acid with a particle having immobilized thereto an oligonucleotide probe comprising a nucleic acid sequence complementary to at least a portion of the target nucleic acid, wherein the oligonucleotide probe comprises a second compound capable of fluorescence and the particle or the oligonucleotide probe further comprises a fluorescence quenching compound in sufficient proximity to said second compound to quench the fluorescence of said second compound prior to hybridization of the oligonucleotide probe to the target nucleic acid, and wherein the particle is capable of scattering electromagnetic radiation of wavelength greater than or equal to about 200 nm; and
c) determining the presence of the authentication code by detecting the scattered electromagnetic radiation of the particle.

27. A nucleic acid-based product authentication kit for detecting an authentication code comprising target nucleic acid, wherein said kit comprises in the same or separate containers:
1) particles
a) capable of scattering electromagnetic radiation of wavelength greater than or equal to about 200 nm, or
b) comprising a compound or plurality of compounds capable of producing a fluorescence signal corresponding to the particle, or
c) capable of scattering electromagnetic radiation of wavelength greater than or equal to about 200 nm and comprising a compound or plurality of compounds capable of producing a fluorescence signal corresponding to the particle; and
2) an oligonucleotide comprising a nucleic acid sequence complementary to at least a portion of the target nucleic acid.

28. The nucleic acid-based product authentication kit of claim 27, further comprising a compound which is capable of binding to duplex nucleic acid and which upon binding or being bound thereto is capable of producing a detectable signal.

29. The nucleic acid-based authentication kit of claim 27, wherein the oligonucleotide comprises a ligand, and wherein the kit further comprises:
a single-strand specific endonuclease; and
a compound capable of fluorescence and capable of binding to the ligand.

30. The nucleic acid-based authentication kit of claim 27, further comprising:
a first compound capable of fluorescence; and
a second compound capable of quenching the fluorescence of the first compound.
